(19) United States
(12) Patent Application Publication
(10) Pub. No.: US 2009/0087900 A1
(43) Pub. Date: Apr. 2, 2009

(54) Apparatus for Performing Electrodistention on Algae Cells

(76) Inventors: Kent Davey, Austin, TX (US); Robert E. Hebner, Austin, TX (US)

Correspondence Address: DILLON & YUDELL LLP, 8911 NORTH CAPITAL OF TEXAS HWY, SUITE 2110, AUSTIN, TX, 78759, US

(21) Appl. No.: 12/233,742

(22) Filed: Sep. 19, 2008

Related U.S. Application Data
(60) Provisional application No. 60/976,036, filed on Sep. 28, 2007.

(51) Int. Cl. C12M 1/42

(52) U.S. Cl. 435/285.2

(57) ABSTRACT

Two apparatuses capable of performing electroporation are disclosed. The first apparatus uses a Marx generator with a substantial change from its original waveform. The second apparatus does not use a Marx generator.

### PRIORITY CLAIM

The present application claims priority under 35 U.S.C. § 119(e)(1) to provisional application No. 60/976,036 filed on Sep. 28, 2007, the contents of which are incorporated herein by reference.

### BACKGROUND OF THE INVENTION

1. Technical Field

The present invention relates to electromechanical manipulation of biological cells in general, and, in particular, to an apparatus for performing electrodistention on algae cells.

2. Description of Related Art

Present electromechanical manipulation of cells generally focuses on one single area of electroporation, which is the usage of an electric field to produce a small hole in a cell wall. The most common application of electroporation is to produce a cell wall hole that is capable of resealing after being used to introduce new material inside of the cell. In addition, multiple electric field pulses can be applied to allow the cell wall hole to remain open for assisting in the extraction of materials from the cell or to cause cell death.

Current researchers discover that electroporation is only one manifestation of a class of effects resulting from electromechanical manipulation of a cell wall. Researches also show that a cell membrane can be stretched in reaction to the interaction force between induced surface charges on a cell membrane and an applied electric field. This mechanical force arises primarily from interfacial charges and commensurate forces on the inner membrane interface. Thus, the point mechanical failure of a cell can be related to its elastic modulus. Under this model, the process of cell membrane disruption is not classical electroporation, but a different electromechanical effect. Using the best data available, the physics behind this electromechanical mechanism predict voltage levels and times that are consistent with experimental results.

### SUMMARY OF THE INVENTION

The present disclosure provides a class of apparatuses for performing electrodistention on cells in order to enhance the extraction of materials from the inside of a cell wall. In accordance with a preferred embodiment of the present invention, an apparatus for performing electrodistention includes a high-voltage, low-current pulse generator constructed with highly reliable parts for industrial use. The apparatus is used for batch or continuous flow of cells in the appropriate growth medium. The apparatus design is determined by the electromechanics of the cell walls and the quantity and flow rate of the material being processed.

All features and advantages of the present invention will become apparent in the following detailed written description.

### BRIEF DESCRIPTION OF THE DRAWINGS

The invention itself, as well as a preferred mode of use, further objects, and advantages thereof, will best be understood by reference to the following detailed description of an illustrative embodiment when read in conjunction with the accompanying drawings, wherein:

FIG. 1 is a diagram of a Marx generator having a shorting spark gap for performing electrodistention;

FIG. 2 is a circuit diagram of the Marx generator from FIG. 1, in accordance with a preferred embodiment of the present invention;

FIG. 3 is an example graph of voltage versus time for a particular application;

FIG. 4 is a diagram of a cable pulse device for performing electrodistention; and

FIG. 5 is a layout of a diffusion plant in which a preferred embodiment of the present invention can be implemented.

### DETAILED DESCRIPTION OF A PREFERRED EMBODIMENT

Two apparatuses capable of performing electroporation in a commercial setting are explained. The first apparatus uses a Marx generator, but with a substantial change to its original waveform. The second apparatus does not use a Marx generator.

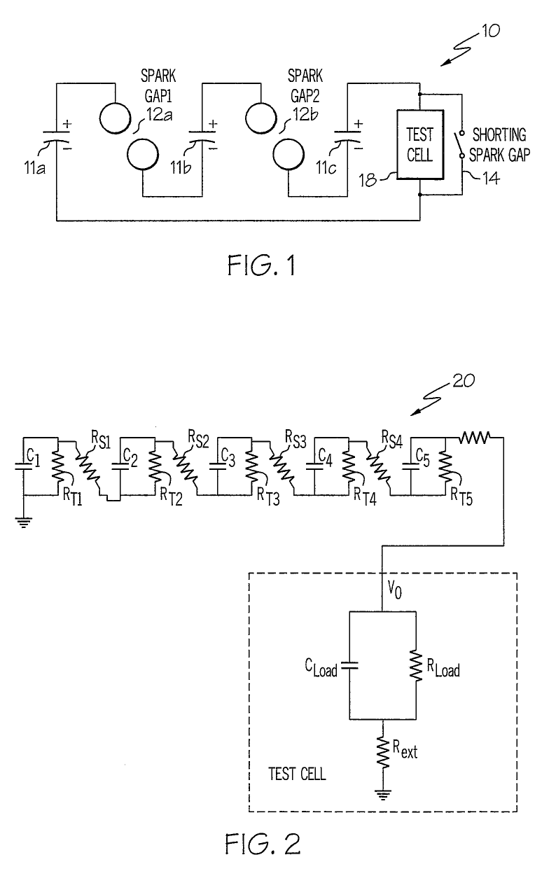

Referring now to the drawings, and in particular to FIG. 1, there is illustrated a diagram of a Marx generator having a shorting spark gap for performing electrodistention, in accordance with a preferred embodiment of the present invention. As shown, a Marx generator 10 includes capacitors 11a-11c, spark gaps 12a-12b and a shorting spark gap 14. The time constant of Marx generator 10 is dictated by the resistance and capacitance of its circuit components. Much of the impedance is presented by a test cell 18 itself. Shorting spark gap 14 can be placed across test cell 18, and can be set to “fire” or discharge when the electric field reaches a specific fraction of its peak. At such point, the electric field will drop in fractions of a microsecond. Higher frequency, shorter pulse width devices may help reduce power dissipation. The configuration of Marx generator 10 is chosen to minimize the energy used to accomplish the electromechanical cell manipulation at a higher frequency, albeit with higher electric fields. Marx generator 10 can be used to perform electromechanical manipulations on algae, sugar cane, and soy beans, as well as materials having related cell structures.

As an alternative embodiment, spark gaps 12a-12b of Marx generator 10 can be replaced by a set of semiconductor switches. The alternative embodiment requires the simultaneous design of test chambers and generators to produce an apparatus that can be achievable with commercially available semiconductor switches. The design process requires the development of response data using Marx generator 10 in FIG. 1 to find the proper parameters for the particular solid state generator in each application.

With reference now to FIG. 2, there is depicted a circuit diagram of a Marx generator, such as Marx generator 10 from FIG. 1, in accordance with a preferred embodiment of the present invention. As shown, a Marx generator 20 includes capacitors C₁, C₂, C₃, C₄, C₅ connected in parallel with resistors R_{T1}, R_{T2}, R_{T3}, R_{T4}, R_{T5}, respectively. In addition, Marx generator 20 also includes a resistor Rₛ₁ connected between resistor R_{T1} and capacitor C₂, a resistor Rₛ₂ connected between resistor R_{T2} and capacitor C₃, a resistor Rₛ₃ connected between resistor R_{T3} and capacitor C₄, a resistor Rₛ₄ connected between resistor R_{T1} and capacitor C₂, and a resistor Rₛ₅ connected between resistor R_{T5} and a test cell 28. Test cell 28 is represented by a capacitor C_{load} connected in parallel with a resistor R_{load}, and connected in series with a resistor Rₑₓₜ. Capacitors C₁-C₅ are charged in parallel and discharged in series. Resistors R_{T1}-R_{T5} and R_{S1}-R_{S5} dictate the rise time and fall times of a pulse to test cell 28. Preferably, C₁=525.5 nF, C₂=529.1 nF, C₃=593.0 nF, C₄=529.7 nF, C₅=564.3 nF, R_{T1}=99Ω, R_{T2}=100.1Ω, R_{T3}=99.9Ω, R_{T4}=300.3Ω, R_{T5}=303.8Ω, Rₛ₁=155.82Ω, Rₛ₂=150.22Ω, Rₛ₃=150.69Ω, Rₛ₄=174.73Ω, Rₛ₅=151.45Ω.

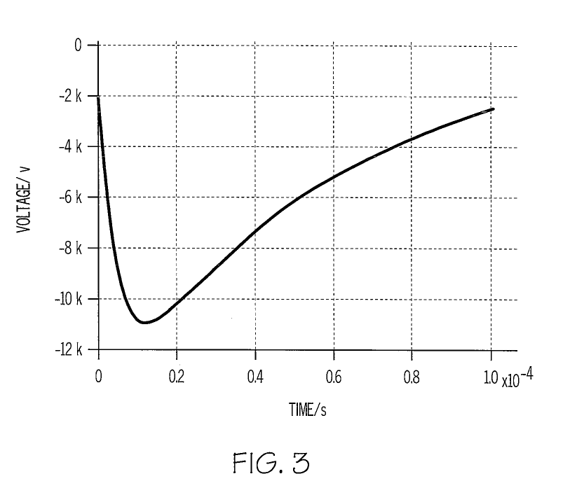

Referring now to FIG. 3, there is depicted an example graph of voltage versus time for Marx generator 20 from FIG. 2. The initial rise time of an electrical pulse is 10-15 us, and the pulse width of the electrical pulse is approximately 100 us. The electric field required for a useful degree of electromechanical manipulations is inversely related to the pulse width of the electrical pulse. Shorter pulse widths will require a greater field strength to accomplish the same effect. A key factor, however, is that for each cell system and transport media, there is an optimal set of pulse parameters that minimize the total energy deposited in order to produce the desired response. Such optimization makes it possible to design appropriate generators for specific applications.

The desired response is impairment or destruction of the cell membrane as well as the cell wall. Without damage to the cell wall, it is difficult to extract lipid molecules that aggregate. Since a cell wall is porous to ions, it is virtually impossible to dielectrically punch a hole in the cell wall. This is to be contrasted with a cell membrane that has a very high resistivity. The underlying thesis for the present disclosure is that electric fields can be used to electromechanically distend a cell to the point that it also damages the cell wall.

Key factors in an efficient system are the minimization of the internal resistance and appropriately matching the impedance of the test system and the pulse generator. In addition, maintenance and life cycle costs are minimized if there are a minimum of components with significant aging issues.

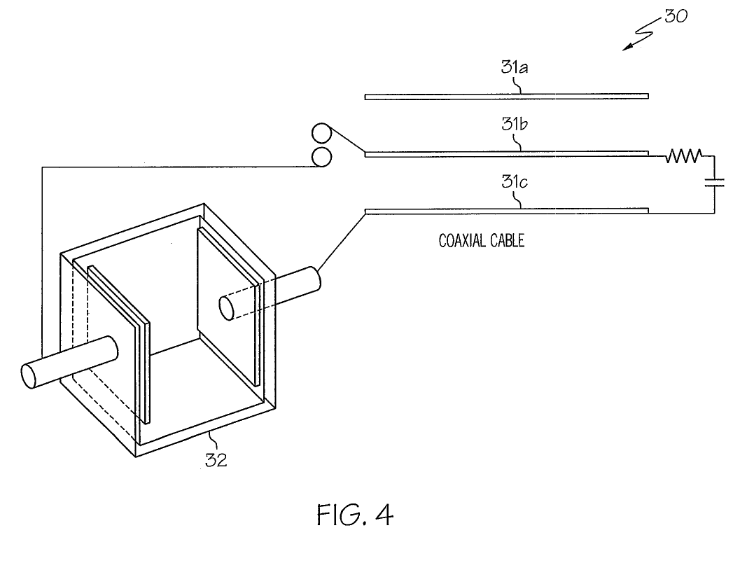

With reference now to FIG. 4, there is illustrated a diagram of a cable pulse device for replacing a Marx generator 10A, such as Marx generator 10 from FIG. 1, to perform electrodistention, in accordance with a preferred embodiment of the present invention. As shown, a cable pulse device 30 includes multiple coaxial cables 31a-31c connecting to a chamber 32. Chamber 32 has a size of approximately 4 inches long, 3″ wide, and 3″ high. Materials intended to be electrodistented are placed inside chamber 21 width for pulses generated by cable pulse device 30 that can be set to equal the length of coaxial cables 31a-31c divided by the speed of light. Coaxial cables 31a-31c can also act as capacitors for storing energy. A relatively short pulse width can be achieved since the capacitance of coaxial cables 31a-31c can be made relatively small.

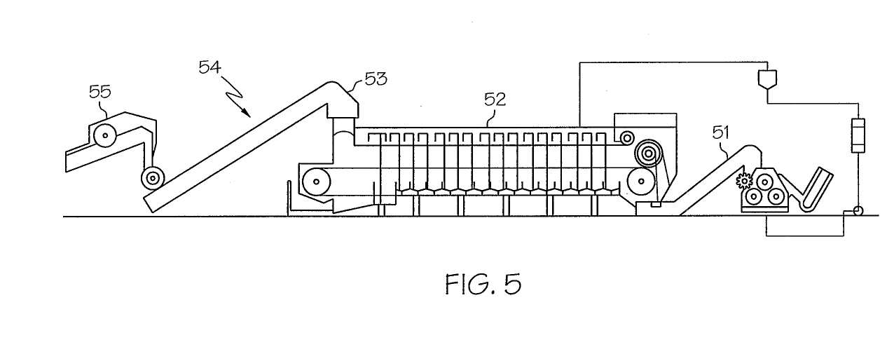

Referring now to FIG. 5, there is illustrated a layout of a diffusion plant in which a preferred embodiment of the present invention can be implemented. As shown, a diffusion plant includes a dewatering equipment 51, a diffuser 52, a feeding equipment 53, a conveyor equipment 54 and a preparation equipment 55. Cane billets are first shredded and dumped onto conveyor equipment 54. They are translated along a washing aquarium for several hundred yards. Water is continuously sprayed over the shredded mat and allowed to percolate through the material. The sugar diffuses from the shredded cane into the water. A typical residence time within the diffusion tank is about six hours. Unlike algae cells, it is only necessary to permanently puncture the sugar cane cell membrane. To the extent that water can get to the sugar cane, small glucose molecules can easily exit the cell. Shredding the sugar cane helps since it allows greater water access. The electromechanical pulsing occurs after the shredder.

The electromechanical manipulation of the cells in sugar cane has the potential to cut the residence time from 6 hours to one hour based on laboratory testing. The electric field is established at the appropriate magnitude and for the appropriate time between the side walls of the first chamber. A one meter wide tank will likely require 400-500 kV pulses.

As has been described, the present invention provides two apparatuses for performing electromechanical manipulations of cells. Such manipulation leads to tearing, stretching, and/or puncture of cells. An indicator of larger scale cell wall destruction has been recorded visually and inferred from the degree of lipid production. The time for the process is quite difficult to determine because the electric stress grows very rapidly, but it is believed to be between 50 and 200 us.

While the invention has been particularly shown and described with reference to a preferred embodiment, it will be understood by those skilled in the art that various changes in form and detail may be made therein without departing from the spirit and scope of the invention.

What is claimed is:

1. An apparatus for performing electrodistention, said apparatus comprising:
a plurality of capacitors;
a plurality of spark gaps connected in series with said plurality of capacitors; and
a shorting spark gap at which a test cell is placed, wherein said shorting spark gap, which is also connected in series with said plurality of capacitors, is set to discharge when an electric field reaches a fraction of its peak.

2. The apparatus of claim 1, wherein said plurality of spark gaps and said plurality of capacitors are connected in an interleave fashion.

3. An apparatus for performing electrodistention, said apparatus comprising:
an electroporation chamber; and
a cable pulse device having a plurality of coaxial cables, coupled to said electroporation chamber, for generating electric pulses to said electroporation chamber to perform electrodistention on materials located within said electroporation chamber.

4. The apparatus of claim 3, wherein pulse widths of electric pulses generated by said cable pulse device are set to equal the length of said plurality of coaxial cables divided by the speed of light.

5. The apparatus of claim 3, wherein said plurality of coaxial cables act as capacitors for storing energy.
